SEQUENCE LISTING (16 sequences)

SEQ ID NO: 1 - protein, 109 aa, Homo Sapiens
EIVMTQSPATLSVSPGERATLSCWASQSISSNLAWYQQKPGQAPRLLIYGASTRATGIPARFSGSGSGTEFTLTISSLQSEDFAIYHCQQYNSWPPLTFGGGTKVEIKR

SEQ ID NO: 2 - protein, 412 aa, Homo Sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 3 - protein, 236 aa, Homo Sapiens
MDMRVPAQLLGLLLLWLRGARCDIQMTQSPSSLSASVGDRVTITCRASQGIRNDLGWYQQKPGKAPKLLIYDASSLESGVPSRFSGSGSGTDFTLTISSLQPEDFATYYCQQFNNYPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 339 aa, Homo Sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 5 - protein, 116 aa, Homo Sapiens
DIQMTQSPSTLSASVGDRVTITCRASQSISSWLAWYQQKPGKAPKLLIYKASSLESGVPSRFSGSGSGTEFTLTISSLQPDDFATYYCQQYNSIEGTFGQGTKVEIKRTVAAPSVF

SEQ ID NO: 6 - protein, 335 aa, Homo Sapiens
MWATLPLLCAGAWLLGVPVCGAAELCVNSLEKFHFKSWMSKHRKTYSTEEYHHRLQTFASNWRKINAHNNGNHTFKMALNQFSDMSFAEIKHKYLWSEPQNCSATKSNYLRGTGPYPPSVDWRKKGNFVSPVKNQGACGSCWTFSTTGALESAIAIATGKMLSLAEQQLVDCAQDFNNHGCQGGLPSQAFEYILYNKGIMGEDTYPYQGKDGYCKFQPGKAIGFVKDVANITIYDEEAMVEAVALYNPVSFAFEVTQDFMMYRTGIYSSTSCHKTPDKVNHAVLAVGYGEKNGIPYWIVKNSWGPQWGMNGYFLIERGKNMCGLAACASYPIPLV

SEQ ID NO: 7 - protein, 1005 aa, Homo Sapiens [sequence omitted]

SEQ ID NO: 8 - protein, 731 aa, Homo Sapiens
MVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 9 - protein, 108 aa, Homo Sapiens
DIQMTQSPSSLSASVGDRVTITCQASQDISIFLNWYQQKPGKAPKLLIYDASKLEAGVPSRFSGTGSGTDFTFTISSLQPEDIATYYCQQFDNLPLTFGGGTKVDFKR

SEQ ID NO: 10 - protein, 782 aa, Homo Sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 11 - protein, 1271 aa, Homo Sapiens
MEALRNPMPLGSSEEALGDLACSSLTGASRDLGTGAVASGTQEETSGPRGDPQQTPSLEKERHTPSRTGPGAAGRTLPRRSRSWERAPRSSRGAQAAACHTSHHSAGSRPGGHLGGQAVGTPNCVPVEGPGCTKEEDVLASSACVSTDGGSLHCHNPSGPSDVPARQPHPEQEGWPPGTGDFPSQVPKQVLDVSQELLQSGVVTLPGTRDRHGRAVVQVRTRSLLWTREHSSCAELTRLLLYFHSIPRKEVRDLGLVVLVDARRSPAAPAVSQALSGLQNNTSPIIHSILLLVDKESAFRPDKDAIIQCEVVSSLKAVHKFVDSCQLTADLDGSFPYSHGDWICFRQRLEHFAANCEEAIIFLQNSFCSLNTHRTPRTAQEVAELIDQHETMMKLVLEDPLLVSLRLEGGTVLARLRREELGTEDSRDTLEAATSLYDRVDEEVHRLVLTSNNRLQQLEHLRELASLLEGNDQQSCQKGLQLAKENPQRTEEMVQDFRRGLSAVVSQAECREGELARWTRSSELCETVSSWMGPLDPEACPSSPVAECLRSCHQEATSVAAEAFPGAGVAVLKPHALGKPWASQQDLWLQYPQTRLRLEEALSEAAPDPSLPPLAQSPPKHERAQEAMRRHQKPPSFPSTDSGGGAWEPAQPLSGLPGRALLCGQDGEPLGPGLCALWDPLSLLRGLPGAGATTAHLEDSSACSSEPTQTLASRPRKHPQKKMIKKTQSFEIPQPDSGPRDSCQPDHTSVFSKGLEVTSTVATEKKLPLWQHARSPPVTQSRSLSSPSGLHPAEEDGRQQVGSSRLRHIMAEMIATEREYIRCLGYVIDNYFPEMERMDLPQGLRGKHHVIFGNLEKLHDFHQQHFLRELERCQHCPLAVGRSFLRHEEQFGMYVIYSKNKPQSDALLSSHGNAFFKDKQRELGDKMDLASYLLRPVQRVAKYALLLQDLLKEASCGLAQGQELGELRAAEVVVCFQLRHGNDLLAMDAIRGCDVNLKEQGQLRCRDEFIVCCGRKKYLRHVFLFEDLILFSKTQKVEGSHDVYLYKQSFKTAEIGMTENVGDSGLRFEIWFRRRQKSQDTYILQASSAEVKSAWTDVIGRILWRQALKSRELRIQEMASMGIGNQPFMDVKPRDRTPDCAVISDGAPKCAVMSDRVPDSIVKGTESQMRGSTAVSSSDHAAPFKRPHSTISDSSTSSSSSQSSSILGSLGLLVSSSPAHPGLWSPAHSPWSSDIRACVEEDEPEPELETGTQAAVCEGAPAVLLSRTRQA

SEQ ID NO: 12 - protein, 584 aa, Homo Sapiens
MTKNPKVNAIKTKALDALGHQYEEAASFQYMRTQSEHQSPTSLLLNRSDGEKHIKTCYEMGGNQTSITEFLLLGFPIGPRIQMLLFGLFSLFYIFILLGNGTILGLISLDSRLHTPMYFFLSHLAVVDIACACSTVPQMLVNLLHPAKPISFAGCMTQMFLFLSFAHTECLLLVVMSYDRYVAICHPLRYSTIMTWKVCITLALTSWILGVLLALVHLVLLLPLSFCGPQKLNHFFCEIMAVLKLACADTHINEVMVLAGAVSVLVGAFFSTVISYVHILCAILKIQSGEGCQKAFSICSSHLCVVGLFYGTAIIMYVEPQYESPKEQKKYLLLFHSLFNPMLNPLIYSLRNKEVQENSEVQNKVLNFVMFTVQNQCLILVVMSYDRYVAICHPFQYTVIMSWRVCTILVLTSWSCGFALSLVHEILLLRLPFCGPRDVNHLFCEILSVLKLACADTWVNQVVIFATCVFVLVGPLSLILVSYMHILGAILKIQTKEGRIKAFSTCSSHLCVVGLFFGIAMVVYMVPDSNQREEQEKMLSLFHSVFNPMLNPLIYSLRNAQLKGALHRALQRKRSMRTVYGLCL

SEQ ID NO: 13 - protein, 1101 aa, Homo Sapiens
MDRAPTEQNVKLSAEVEPFIPQKKSPDTFMIPMALPNDNGSVSGVEPTPIPSYLITCYPFVQENQSNRQFPLYNNDIRWQQPNPNPTGPYFAYPIISAQPPVSTEYTYYQLMPAPCAQVMGFYHPFPTPYSNTFQAANTVNAITTECTERPSQLGQVFPLSSHRSRNSNRGSVVPKQQLLQQHIKSKRPLVKNVATQKETNAAGPDSRSKIVLLVDASQQTDFPSDIANKSLSETTATMLWKSKGRRRRASHPTAESSSEQGASEADIDSDSGYCSPKHSNNQPAAGALRNPDSGTMNHVESSMCAGGVNWSNVTCQATQKKPWMEKNQTFSRGGRQTEQRNNSQVGFRCRGHSTSSERRQNLQKRPDNKHLSSSQSHRSDPNSESLYFEDEDGFQELNENGNAKDENIQQKLSSKVLDDLPENSPINIVQTPIPITTSVPKRAKSQKKKALAAALATAQEYSEISMEQKKLQEALSKAAGKKNKTPVQLDLGDMLAALEKQQQAMKARQITNTRPLSYTVVTAASFHTKDSTNRKPLTKSQPCLTSFNSVDIASSKAKKGKEKEIAKLKRPTALKKVILKEREEKKGRLTVDHNLLGSEEPTEMHLDFIDDLPQEIVSQEDTGLSMPSDTSLSPASQNSPYCMTPVSQGSPASSGIGSPMASSTITKIHSKRFREYCNQVLCKEIDECVTLLLQELVSFQERIYQKDPVRAKARRRLVMGLREVTKHMKLNKIKCVIISPNCEKIQSKGGLDEALYNVIAMAREQEIPFVFALGRKALGRCVNKLVPVSVVGIFNYFGAESLFNKLVELTEEARKAYKDMVAAMEQEQAEEALKNVKKVPHHMGHSRNPSAASAISFCSVISEPISEVNEKEYETNWRNMVETSDGLEASENEKEVSCKHSTSEKPSKLPFDTPPIGKQPSLVATGSTTSATSAGKSTASDKEEVKPDDLEWASQQSTETGSLDGSCRDLLNSSITSTTSTLVPGMLEEEEDEDEEEEEDYTHEPISVEVQLNSRIESWVSETQRTMETLQLGKTLNGSEEDNVEQSGEEEAEAPEVLEPGMDSEAWTADQQASPGQQKSSNCSSLNKEHSDSNYTTQTT

SEQ ID NO: 14 - protein, 108 aa, Homo Sapiens
ZIVLTZSPGTLSLSPGZRAALSCRASQSLSGNYLAWYQQKPGQAPRLLMYGVSSRATGIPDRFSGSGSGADFTLTISRLZPEDFAVYYCQQYGSSPFTFGQGSKLEIK

SEQ ID NO: 15 - protein, 118 aa, Homo Sapiens
EIVLTQSPATLSLSPGERASLSCRASQSVATYLAWYQHKPGQAPRLLIYDASNRATGIPARFSGSGSGTDFTLTISSLEPADFAVYYCQHRNNWPPLFTFGPGTRVDVKRTVAAPSVF

SEQ ID NO: 16 - protein, 1056 aa, Homo Sapiens
  misc_feature (1)..(1): Xaa can be any naturally occurring amino acid
XFKMQDYNKDDMSYRRISAVEPKTALPFNRFLPNKSRQPSYVPAPLRKKKPDKHEDNRRSWASPVYTEADGTFSSQFLLLQALQTYSDDILSSETHTKIDPTSGPRLITRRKNLSYAPGYRRDDLEMAALDPDLENDDFFVRKTGAFHANPYVLRAFEDFRKFSEQDDSVERDIILQCREGELVLPDLEKDDMIVRRIPAQKKEVPLSGAPDRYHPVPFPEPWTLPPEIQAKFLCVLERTCPSKEKSNSCRILVPSYRQKKDDMLTRKIQSWKLGTTVPPISFTPGPCSEADLKRWEAIREASRLRHKKRLMVERLFQKIYGENGSKSMSDVSAEDVQNLRQLRYEEMQKIKSQLKEQDQKWQDDLAKWKDRRKSYTSDLQKKKEEREEIEKQALEKSKRSSKTFKEMLQDRESQNQKSTVPSRRRMYSFDDVLEEGKRPPTMTVSEASYQSERVEEKGATYPSEIPKEDSTTFAKREDRVTTEIQLPSQSPVEEQSPASLSSLRSRSTQMESTRVSASLPRSYRKTDTVRLTSVVTPRPFGSQTRGISSLPRSYTMDDAWKYNGDVEDIKRTPNNVVSTPAPSPDASQLASSLSSQKEVAATEEDVTRLPSPTSPFSSLSQDQAATSKATLSSTSGLDLMSESGEGEISPQREVSRSQDQFSDMRISINQTPGKSLDFGFTIKWDIPGIFVASVEAGSPAEFSQLQVDDEIIAINNTKFSYNDSKEWEEAMAKAQETGHLVMDVRRYGKAGSPETKWIDATSGIYNSEKSSNLSVTTDFSESLQSSNIESKEINGIHDESNAFESKASESISLKNLKRRSQFFEQGSSDSVVPDLPVPTISAPSRWVWDQEEERKRQERWQKEQDRLLQEKYQREQEKLREEWQRAKQEAERENSKYLDEELMVLSSNSMSLTTREPSLATWEATWSEGSKSSDREGTRAGEEERRQPQEEVVHEDQGKKPQDQLVIERERKWEQQLQEEQEQKRLQAEAEEQKRPAEEQKRQAEIERETSVRIYQYRRPVDSYDIPKTEEASSGFLPGDRNKSRSTTELDDYST

(19) United States
(12) Patent Application Publication
(10) Pub. No.: US 2017/0248609 A1
(43) Pub. Date: Aug. 31, 2017

(54) BIOMARKERS PREDICTIVE OF MUSCLE ATROPHY, METHOD AND USE

(71) Applicants: Stefan REINKER, Basel (CH); Ronenn ROUBENOFF, Cambridge, MA (US); Yingqi Karen WANG, Boxborough, MA (US); Kan ZHU, Cambridge, MA (US)

(72) Inventors: Stefan REINKER, Basel (CH); Ronenn ROUBENOFF, Cambridge, MA (US); Yingqi Karen WANG, Boxborough, MA (US); Kan ZHU, Cambridge, MA (US)

(73) Assignee: Novartis AG, Basel (CH)

(21) Appl. No.: 15/114,248

(22) PCT Filed: Jan. 26, 2015
(86) PCT No.: PCT/IB2015/050561

§ 371 Date: Jul. 26, 2016

Related U.S. Application Data
(60) Provisional application No. 61/931,932, filed on Jan. 27, 2014.

(51) Int. Cl. G01N 33/68

(52) U.S. Cl. CPC G01N 33/6887; G01N 33/6893; G01N 2800/10; G01N 2800/52; G01N 2800/50

(57) ABSTRACT

This invention is in the field of Biomarkers. In particular, it relates to biomarker being predictive of muscle atrophy. The invention relates to the use of such biomarker for selectively treating a patient having muscle atrophy and a method of predicting the likelihood that a patient having muscle atrophy will respond to treatment with a muscle anabolic agent.

### BACKGROUND OF THE DISCLOSURE

Human skeletal muscle is composed of muscle fibers that are classified depending on their speed of contraction and predominant type of energy metabolism. Muscle fibers can be classified as type I (slow-twitch) and type II (fast-twitch) fibers based on their predominant myosin heavy chain (MyHC) isoform content. Generally, type I and type IIa fibers utilize oxidative phosphorylation, whereas type IIx and IIb fibers harness anaerobic metabolism to generate ATP. Both the percentage and structural morphology of the fiber type will determine the phenotypic capacity and functional performance of any given muscle. In addition to genetic background, environmental factors in both health and disease have a direct impact leading to changes in fiber type/morphology and consequent functionality; such processes include aging, exercise, diabetes, disuse atrophy, chronic heart failure, and muscle atrophy.

Examples of conditions with a muscle atrophy component are disuse atrophy, cachexia, sporadic inclusion body myositis, renal failure, AIDS (acquired immunodeficiency syndrome), cancer, heart failure, and COPD (chronic obstructive pulmonary disease).

Cachexia is generally considered an acquired, accelerated loss of muscle caused by an underlying disease (Evans W J, Morley J E, Angles J, et al. Cachexia: a new definition. Clinical Nutrition 2008; 27: 793-799). When cachexia is seen in a patient with end-stage cancer, it is called “cancer cachexia”. Cancer cachexia affects the majority of patients with advanced cancer and is associated with a reduction in treatment tolerance, response to therapy, quality of life and duration of survival. Cancer cachexia has been defined recently as a multifactorial syndrome characterized by an ongoing loss of skeletal muscle mass, with or without loss of fat mass, which cannot be fully reversed by conventional nutritional support and leads to progressive functional impairment. Skeletal muscle loss appears to be the most significant event in cancer cachexia. The international consensus on the classification of cancer cachexia suggested that diagnostic criteria should take into account not only that weight loss is a signal event of the cachectic process but that the initial reserve of the patient should also be considered, either as low BMI or as low level of muscularity. Although the latter concept has some validation in terms of clinical risk, there is thus a need for methods enabling evaluation of the biological correlates in terms of changes within skeletal muscle itself.

However, the precise molecular mechanisms that mediate the changes in protein synthesis and degradation that ultimately lead to atrophy of muscle fibers, such as in cancer cachexia, are not known. For each animal model that has been studied, different pathways have been implicated. Also, human data are very limited.

One report of specific urinary indicators of cancer cachexia was published by Skipworth et al.: Mass spectrum detection of candidate protein biomarkers of cancer cachexia in human urine. International Journal of Oncology 36: 973-982, 2010. However, since the molecular mechanism is not known, there is a great risk of false positives. Furthermore, only a qualitative determination of indications is provided. Thus, there is a need of alternative biomarkers enabling an early diagnosis of atrophy of muscle fibers, such as in cachexia.

### BRIEF DESCRIPTION OF THE DISCLOSURE

This present invention describes a method of identifying individuals who are likely to develop a condition where they suffer from muscle atrophy, such as cachexia, before they experience symptoms or reduced physical functioning. Since cachexia is a common complication of cancer, which increases cancer mortality, the method may be particularly advantageous for identifying cancer patients who are likely to develop cachexia. The invention utilizes one or more of a panel of protein markers to diagnose muscle atrophy in a patient, such as in cancer cachexia. This is based on a urine sample from the individual, independently of whether the individual has lost weight or not. This invention may be used to identify patients who would benefit from an anabolic intervention to reverse the muscle atrophy, or to stratify patients on response to therapy, or on need for higher dose of therapy. Particularly, the method allows for quantitative analysis of patients.

According to a first aspect, a method of selectively treating muscle atrophy in a patient is provided. The method comprises selectively administering a therapeutically effective amount of a muscle anabolic agent to the patient on the basis of the patient having significantly increased levels of the polypeptides in the group consisting of SEQ ID NOs 1 to 16, in urine.

According to a second aspect, a method of selectively treating a patient having muscle atrophy with a muscle anabolic agent is provided. The method comprises a first step of selecting the patient for treatment with a muscle anabolic agent on the basis of a the patient having significantly increased levels of the polypeptides in the group consisting of SEQ ID NOs 1 to 16, in urine; and a second step of administering a therapeutically effective amount of a muscle anabolic agent to the patient.

According to a third aspect, a method of selectively treating a patient having muscle atrophy with a muscle anabolic agent is provided. The method comprises a first step of assaying a urine sample from the patient for the polypeptides in the group consisting of SEQ ID NOs 1 to 16; a second step of selecting the patient for treatment with a muscle anabolic agent on the basis of the urine sample from the patient having significantly increased levels of the polypeptides in the group consisting of SEQ ID NOs 1 to 16; and a third step of administering a therapeutically effective amount of a muscle anabolic agent to the patient.

According to a fourth aspect, a method of predicting the likelihood that a patient having muscle atrophy will respond to treatment with a muscle anabolic agent is provided. The method comprises assaying a urine sample from the patient for the presence or absence of the polypeptides in the group consisting of SEQ ID NOs 1 to 16, wherein. A significantly increased levels of the polypeptides in the group consisting of SEQ ID NOs 1 to 16 in urine is indicative of an increased likelihood that the patient will respond to treatment with a muscle anabolic agent; and the absence of significantly increased levels of the polypeptides in the group consisting of SEQ ID NOs 1 to 16 in urine is indicative of a decreased likelihood that the patient will respond to treatment with a muscle anabolic agent.

According to a fifth aspect, a method for producing a transmittable form of information for predicting the responsiveness of a patient having muscle atrophy to treatment with a muscle anabolic agent is provided. The method comprises determining an increased likelihood of the patient responding to treatment with the a muscle anabolic agent based on significantly increased levels of the polypeptides in the group consisting of SEQ ID NOs 1 to 16, in urine; and recording the result of the determining step on a tangible or intangible media form for use in transmission.

According to a sixth aspect, a method of predicting the likelihood that a patient having muscle atrophy will respond to treatment with a muscle anabolic agent is provided. The method comprises a first step of providing a urine sample from the patient; a second step of assaying a urine sample from the patient for levels of the polypeptides in the group consisting of SEQ ID NOs 1 to 16 in the sample, such as by subjecting the sample to LC-MS/MS analysis; and a third step of reporting whether the polypeptides in the group consisting of SEQ ID NOs 1 to 16 in urine is present in significantly increased levels in the sample, wherein the significantly increased levels of the polypeptides in the group consisting of SEQ ID NOs 1 to 16 in urine is indicative of an increased likelihood of response to a muscle anabolic agent.

According to a seventh aspect, a method of selectively predicting development of muscle atrophy in a patient is provided, comprising identifying a patient on the basis of the patient having significantly increased levels of the polypeptides in the group consisting of SEQ ID NOs 1 to 16, in urine, wherein significantly increased levels of the polypeptides in the group consisting of SEQ ID NOs 1 to 16 in urine is indicative of a predisposition for developing muscle atrophy.

According to a eight aspect, a method of selectively predicting development of muscle atrophy in a patient is provided, comprising: assaying a urine sample from the patient for the polypeptides in the group consisting of SEQ ID NOs 1 to 16; thereafter, selecting the patient for treatment with a muscle anabolic agent on the basis of the urine sample from the patient having significantly increased levels of the polypeptides in the group consisting of SEQ ID NOs 1 to 16.

According to an ninth aspect, a method of predicting the likelihood that a patient will develop muscle atrophy is provided, comprising assaying a urine sample from the patient for the presence or absence of the polypeptides in the group consisting of SEQ ID NOs 1 to 16, wherein: significantly increased levels of the polypeptides in the group consisting of SEQ ID NOs 1 to 16 in urine is indicative of an increased likelihood that the patient will develop muscle atrophy; and the absence of significantly increased levels of the polypeptides in the group consisting of SEQ ID NOs 1 to 16 in urine is indicative of a decreased likelihood that the patient will develop muscle atrophy.

According to a tenth aspect, a method for producing a transmittable form of information for predicting the if a patient will develop muscle atrophy is provided, comprising: determining an increased likelihood of the patient developing muscle atrophy based on significantly increased levels of the polypeptides in the group consisting of SEQ ID NOs 1 to 16, in urine; and recording the result of the determining step on a tangible or intangible media form for use in transmission.

According to a eleventh aspect, a method of predicting the likelihood that a patient will develop muscle atrophy is provided, comprising: providing a urine sample from the patient; assaying a urine sample from the patient for levels of the polypeptides in the group consisting of SEQ ID NOs 1 to 16 in the sample, such as by subjecting the sample to LC-MS/MS analysis; and reporting whether the polypeptides in the group consisting of SEQ ID NOs 1 to 16 in urine is present in significantly increased levels in the sample, wherein the significantly increased levels of the polypeptides in the group consisting of SEQ ID NOs 1 to 16 in urine is indicative of an increased likelihood of the patient developing muscle atrophy.

In the methods according to aspects of the invention, the step of assaying may comprise a technique selected from the group consisting of immunoassays, immunohistochemistry, ELISA, flow cytometry, Western blot, HPLC, mass spectrometry, alone or in combination, such as LC-MS/MS.

According to a twelfth aspect, a muscle anabolic agent for use in treating a patient having muscle atrophy is provided, characterized in that a therapeutically effective amount of a muscle anabolic agent is to be administered to the patient on the basis of said patient having significantly increased levels of the polypeptides in the group consisting of SEQ ID NOs 1 to 16, in urine.

According to a thirteenth aspect, a muscle anabolic agent for the use in the treatment of muscle atrophy in a patient is provided, comprising assaying a urine sample from the patient, determining if a patient is having significantly increased levels of the polypeptides in the group consisting of SEQ ID NOs 1 to 16, in urine, and if elevated levels of the polypeptides in the group consisting of SEQ ID NOs 1 to 16 is present, a therapeutically effective amount of a muscle anabolic agent is to be administered to the patient.

According to an fourteenth aspect, a muscle anabolic agent for use in treating a patient having muscle atrophy is provided characterized in that the patient is selected for treatment with the a muscle anabolic agent on the basis of the patient having significantly increased levels of the polypeptides in the group consisting of SEQ ID NOs 1 to 16, in urine; and thereafter, a therapeutically effective amount of a muscle anabolic agent is administered to the patient.

According to a fifteenth aspect, a muscle anabolic agent for use in treating a patient having muscle atrophy is provided, characterized in that a urine sample from the patient is assayed for the polypeptides in the group consisting of SEQ ID NOs 1 to 16; and a therapeutically effective amount of a muscle anabolic agent is selectively administered to the patient on the basis of the urine sample from the patient having significantly increased levels of the polypeptides in the group consisting of SEQ ID NOs 1 to 16.

According to a sixteenth aspect, a muscle anabolic agent for use in treating a patient having muscle atrophy is provided, characterized in that a urine sample from the patient is assayed for the polypeptides in the group consisting of SEQ ID NOs 1 to 16; the patient is selected for treatment with a muscle anabolic agent on the basis of the urine sample from the patient having significantly increased levels of the polypeptides in the group consisting of SEQ ID NOs 1 to 16; and a therapeutically effective amount of a muscle anabolic agent is to be selectively administered to the patient.

The agent according to aspects of the invention, may be characterized based on the step of assaying comprising a technique selected from the group consisting of immunoassays, immunohistochemistry, ELISA, flow cytometry, Western blot, HPLC, mass spectrometry, alone or in combination, such as LC-MS/MS.

According to an seventeenth aspect, a kit for use in predicting the likelihood that a patient having muscle atrophy will respond to treatment with a muscle anabolic agent is provided comprising, at least one probe capable of detecting the presence of the polypeptides in the group consisting of SEQ ID NOs 1 to 16; and instructions for using the probe to assay a biological sample from the muscle atrophy patient for the presence of the polypeptides in the group consisting of SEQ ID NOs 1 to 16, wherein significantly increased levels of the polypeptides in the group consisting of SEQ ID NOs 1 to 16 is indicative of an increased likelihood that the patient will respond to treatment with the muscle anabolic agent and the absence of significantly increased levels of the polypeptides in the group consisting of SEQ ID NOs 1 to 16 is indicative of a decreased likelihood that the patient will respond to treatment with the muscle anabolic agent.

According to a eighteenth aspect, a kit for use in treating a patient having muscle atrophy is provided comprising, a therapeutically effective amount of a muscle anabolic agent; at least one probe capable of detecting the presence of the polypeptides in the group consisting of SEQ ID NOs 1 to 16; instructions for using the probe to assay a biological sample from the patient for significantly increased levels of the polypeptides in the group consisting of SEQ ID NOs 1 to 16, instructions for administering the muscle anabolic agent to the patient if the biological sample from the patient has significantly increased levels of the polypeptides in the group consisting of SEQ ID NOs 1 to 16; and optionally, means for administering the muscle anabolic agent to the patient.

In methods, muscle anabolic agents or kits according to different aspects, the muscle atrophy may be selected from the group consisting of disuse atrophy, cachexia, sporadic inclusion body myositis, renal failure, AIDS (acquired immunodeficiency syndrome) and COPD (chronic obstructive pulmonary disease). The cachexia may be cancer cachexia, and the cancer may for example be gastrointestinal cancer, pancreatic cancer, or lung cancer.

In methods, muscle anabolic agents or kits according to different aspects, a significant increased level of at least 2, 3, 4, 5, 6, 7, 8, 9 or 10 of the polypeptides in the group consisting of SEQ ID NOs 1 to 16, in urine, may be selected.

In methods, muscle anabolic agents or kits according to different aspects, a significantly increased levels of the polypeptides in the group consisting of SEQ ID NOs 1 to 16, in urine, may be at least 50% above, at least 60% above, at least 70% above, at least 80% above, at least 90% above, at least 100% above, at least 110% above, at least 120% above, at least 130% above, at least 140% above, at least 150% above, at least 160% above, at least 170% above, at least 180% above, at least 190% above, or at least 200% above a standardized range of values.

In methods, muscle anabolic agents or kits according to different aspects, the muscle anabolic agent is an ActRIIB antibody, such as bimagrumab, an ActRIIA antibody, a soluble ActRIIB decoy mimetic, an anti-myostatin antibody, a myostatin propeptide, a myostatin decoy protein that binds ActRIIB but does not activate it, a beta 2 agonist, a Ghrelin agonist, an IGF-1 protein or a mimetic thereof a SARM, GH agonists/mimetics or follistatin.

### BRIEF DESCRIPTION OF THE DRAWINGS

FIG. 1 is a graph showing a volcano plot of the statistical analysis of urine protein intensity vs. previous weight loss by ANOVA (x axis represents slope of association of the urine protein concentration with the weight loss in patient, and y axis represents p-value), with the 16 selected proteins highlighted in black.

### DETAILED DESCRIPTION OF THE DISCLOSURE

The methods, compositions and kits of the present invention therefore provide a means for selecting patients susceptible for muscle atrophy, thereby enhancing the therapeutic efficacy of such treatments.

Therefore, in one aspect, the invention provides a composition comprising muscle anabolic agent for use in the treatment of muscle atrophy in a patient, wherein the patient is selected on the basis of a specific urinary biomarker. In one embodiment, the urinary biomarker for predicting therapeutic responsiveness to therapy is a polypeptide according to SEQ ID NOs 1 to 16. In one embodiment of the disclosure, a significant increase in the level of the polypeptides according to SEQ ID NOs 1 to 16 in the urine of a patient is indicative of an increased likelihood (i) that a patient will develop muscle atrophy or weakness and/or (ii) that the patient will respond to treatment with a muscle anabolic agent. In another embodiment of the disclosure, the significant increased or elevated protein concentration as described herein also refers to a situation in which the significant increase at least one, or at least two, or at least three, or at least five, or at least six, or at least seven, or at least eight, or at least nine, or at least ten, or at least eleven, or at least twelve, or at least thirteen, or at least fourteen, or at least fifteen of the polypeptides according to SEQ ID NOs 1 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 1 and one or more of the proteins selected from the group consisting of the polypeptides SEQ ID NO 2-16 is

indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 2 and one or more of the proteins selected from the group consisting of the polypeptides SEQ ID NO 1 and 3 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 3 and one or more of the proteins selected from the group consisting of the polypeptides SEQ ID NO 1 to 2 and 4 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 4 and one or more of the proteins selected from the group consisting of the polypeptides SEQ ID NO 1 to 3 and 5 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 5 and one or more of the proteins selected from the group consisting of the polypeptides SEQ ID NO 1 to 4 and 6 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 6 and one or more of the proteins selected from the group consisting of the polypeptides SEQ ID NO 1 to 5 and 7 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 7 and one or more of the proteins selected from the group consisting of the polypeptides SEQ ID NO 1 to 6 and 8 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 8 and one or more of the proteins selected from the group consisting of the polypeptides SEQ ID NO 1 to 7 and 9 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 9 and one or more of the proteins selected from the group consisting of the polypeptides SEQ ID NO 1 to 8 and 10 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 10 and one or more of the proteins selected from the group consisting of the polypeptides SEQ ID NO 1 to 9 and 11 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 11 and one or more of the proteins selected from the group consisting of the polypeptides SEQ ID NO 1 to 10 and 12 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 12 and one or more of the proteins selected from the group consisting of the polypeptides SEQ ID NO 1 to 11 and 13 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 13 and one or more of the proteins selected from the group consisting of the polypeptides SEQ ID NO 1 to 12 and 14 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 14 and one or more of the proteins selected from the group consisting of the polypeptides SEQ ID NO 1 to 13 and 15 to 16 is indicative of an increased likelihood (i) that a patient will develop muscle atrophy or weakness and/or (ii) that the patient will respond to treatment with a muscle anabolic agent.

In one embodiment of the disclosure the significant increased or elevated protein concentration of the polypeptide according to SEQ ID NO 15 and one or more of the proteins selected from the group consisting of the polypeptides SEQ ID NO 1 to 14 and 16 is indicative of an increased likelihood (i) that a patient will develop muscle atrophy or weakness and/or (ii) that the patient will respond to treatment with a muscle anabolic agent.

Preferably, the mentioned composition is used as described herein to treat cancer cachexia.

In order that the present invention may be more readily understood, certain terms are first defined. Additional definitions are set forth throughout the detailed description.

The term “comprising” encompasses “including” as well as “consisting,” e.g. a composition “comprising” X may consist exclusively of X or may include something additional, e.g., X+Y.

The term “about” in relation to a numerical value x means +/−10% unless the context dictates otherwise.

The term “biomarker” refers to one or more polypeptides that can be used to: diagnose, or to aid in the diagnosis or prognosis of muscle atrophy either alone or as combination of multiple polypeptides; monitor the progression of muscle atrophy; and/or monitor the effectiveness of a muscle atrophy treatment. Additionally, a “biomarker” also refers to those proteins or polypeptide fragments thereof listed in Table 1 being present and elevated or significantly increased in urine samples taken from subjects having a risk of developing muscle atrophy, weakness or becoming cachexic.

As used herein, the terms “subject” and “patient” include any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

The term “assaying” is used to refer to the act of identifying, screening, probing, testing measuring or determining, which act may be performed by any conventional means. For example, a sample may be assayed for the presence of a particular genetic or protein marker by using an ELISA assay, a Northern blot, imaging, serotyping, cellular typing, gene sequencing, phenotyping, haplotyping, immunohistochemistry, western blot, mass spectrometry, etc.

The term “detecting” (and the like) means the act of extracting particular information from a given source, which may be direct or indirect. In some embodiments of the predictive methods disclosed herein, the presence of a given thing (e.g., allele, level of protein, etc.) is detected in a biological sample indirectly, e.g., by querying a database. The terms “assaying” and “determining” contemplate a transformation of matter, e.g., a transformation of a biological sample, e.g., a blood sample or other tissue sample, from one state to another by means of subjecting that sample to physical testing.

The term “obtaining” means to procure, e.g., to acquire possession of in any way, e.g., by physical intervention (e.g., biopsy, blood draw) or non-physical intervention (e.g, transmittal of information via a server), etc.

The phrase “assaying a biological sample . . . ” and the like, is used to mean that a sample may be tested (either directly or indirectly) for either the presence or the absence of a given atrophy response marker. It will be understood that, in a situation where the presence of a substance denotes one probability and the absence of a substance denotes a different probability, then either the presence or the absence of such substance may be used to guide a therapeutic decision. For example, one may determine if a patient has atrophy response marker by determining the actual existence of particular response allele in the patient or by determining the absence of the particular response allele in the patient. In both such cases, one has determined whether the patient has the presence of the atrophy response marker. The disclosed methods involve, inter alia, determining whether a particular individual has an atrophy response marker.

As used herein, the phrase “atrophy response marker(s)” collectively refers to a marker of predicting muscle atrophy. In some embodiments of the disclosed methods, uses, and kits, the patient has at least one atrophy response marker.

As used herein, the term “treating” or “treatment” of any disease or disorder (i.e., cancer cachexia) refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art.

The term “significant increased level” means a quantitatively increased value, such as an amount, compared to a reference value, such as an amount. Consequently, the terms “significantly increased level” or “elevated level” can refer to an increased/elevated protein concentration compared to a control group of healthy subjects, wherein the concentration of said proteins or polypeptide fragments thereof is considered to be increased/elevated if the amount of said proteins and/or polypeptide fragments thereof in the urine of a subject is at least 50% above, at least 60% above, at least 70% above, at least 80% above, at least 90% above, at least 100% above, at least 110% above, at least 120% above, at least 130% above, at least 140% above, at least 150% above, at least 160% above, at least 170% above, at least 180% above, at least 190% above, or at least 200% above a standardized range of values. The standardized range of values may also be fixed values, i.e. values independent of a particular sample group of individuals.

The term “standardized range of values” refers to the upper limit of a statistically relevant standard deviation calculated on the basis of the average concentration of the herein disclosed biomarker proteins or fragments thereof in a healthy control group.

The term “detect”, “detected” or “detecting” includes measure, measured or measuring.

The term “pharmaceutically acceptable” means a nontoxic material that does not interfere with the effectiveness of the biological activity of the active ingredient(s).

The term “administering” in relation to a compound, e.g., an IL-17 binding molecule or another agent, is used to refer to delivery of that compound to a patient by any route.

As used herein, a “therapeutically effective amount” refers to an amount of an muscle anabolic agent that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., a muscle anabolic agent) administered alone, the term refers to that ingredient alone. When applied to a combination, the term refers to combined amounts of the active ingredients that result in the therapeutic effect, whether administered in combination, serially or simultaneously.

As used herein, “selecting” and “selected” in reference to a patient is used to mean that a particular patient is specifically chosen from a larger group of patients on the basis of (due to) the particular patient having a predetermined criteria, e.g., the patient has an atrophy response marker. Similarly, “selectively treating” refers to providing treatment to a patient having a particular disease, where that patient is specifically chosen from a larger group of patients on the basis of the particular patient having a predetermined criterion, e.g., a cancer patient specifically chosen for treatment due to the patient having an atrophy response marker. Similarly, “selectively administering” refers to administering a drug to a patient that is specifically chosen from a larger group of patients on the basis of (due to) the particular patient having a predetermined criterion, e.g., a particular genetic or other biological marker. By selecting, selectively treating and selectively administering, it is meant that a patient is delivered a personalized therapy based on the patient's particular biology, rather than being delivered a standard treatment regimen based solely on the patient having a particular disease. Selecting, in reference to a method of treatment as used herein, does not refer to fortuitous treatment of a patient that has an atrophy response marker, but rather refers to the deliberate choice to administer a muscle anabolic agent to a patient based on the patient having an atrophy response marker. Thus, selective treatment differs from standard treatment, which delivers a particular drug to all patients, regardless of their allelic status.

As used herein, “predicting” indicates that the methods described herein provide information to enable a health care provider to determine the likelihood that an individual will develop muscle atrophy, or that a patient having an atrophy disease selected from will respond to or will respond more favorably to treatment with a muscle anabolic agent. It does not refer to the ability to predict response with 100% accuracy. Instead, the skilled artisan will understand that it refers to an increased probability.

As used herein, “likelihood” and “likely” is a measurement of how probable an event is to occur. It may be used interchangeably with “probability”. Likelihood refers to a probability that is more than speculation, but less than certainty. Thus, an event is likely if a reasonable person using common sense, training or experience concludes that, given the circumstances, an event is probable. In some embodiments, once likelihood has been ascertained, the patient may be treated (or treatment continued, or treatment proceed with a dosage increase) with the muscle anabolic agent or the patient may not be treated (or treatment discontinued, or treatment proceed with a lowered dose) with the muscle anabolic agent.

The phrase “increased likelihood” refers to an increase in the probability that an event will occur. For example, some methods herein allow prediction of whether a patient will display an increased likelihood of responding to treatment with a muscle anabolic agent or an increased likelihood of responding better to treatment with a muscle anabolic agent in comparison to a patient having an atrophy disease who does not have an atrophy response marker.

The term “biological sample” as used herein refers to a sample from a patient, which may be used for the purpose of identification, diagnosis, prediction, or monitoring. Preferred samples include synovial fluid, blood, blood-derived product (such as buffy coat, serum, and plasma), lymph, urine, tear, saliva, hair bulb cells, cerebrospinal fluid, buccal swabs, feces, synovial fluid, synovial cells, sputum, or tissue samples (e.g., cartilage samples). In addition, one of skill in the art would realize that some samples would be more readily analyzed following a fractionation or purification procedure, for example, isolation of DNA from whole blood.

The term “muscle atrophy” refers to decrease in the mass of the muscle; it can be a partial or complete wasting away of muscle. It includes any kind of muscle atrophy, including as a result of treatment with a glucocorticoid such as cortisol, dexamethasone, betamethasone, prednisone, methylprednisolone, or prednisolone. The muscle atrophy can also be a result of denervation due to nerve trauma or a result of degenerative, metabolic, or inflammatory neuropathy (e.g., Guillian-Barre syndrome, peripheral neuropathy, or exposure to environmental toxins or drugs). In addition, the muscle atrophy can be a result of myopathy, such as myotonia; a congential myopathy, including nemalene myopathy, multi/minicore myopathy and myotubular (centronuclear) myopathy; mitochondrial myopathy; familial periodic paralysis; inflammatory myopathy; metabolic myopathy, such as caused by a glycogen or lipid storage disease; dermatomyositisis; polymyositis; inclusion body myositis; myositis ossificans; rhabdomyolysis and myoglobinurias. The myopathy may be caused by a muscular dystrophy syndrome, such as Duchenne, Becker, myotonic, fascioscapulohumeral, Emery-Dreifuss, oculopharyngeal, scapulohumeral, limb girdle, Fukuyama, a congenital muscular dystrophy, or hereditary distal myopathy. In addition, the muscle atrophy can be a result of an adult motor neuron disease, infantile spinal muscular atrophy, amyotrophic lateral sclerosis, juvenile spinal muscular atrophy, autoimmune motor neuropathy with multifocal conductor block, paralysis due to stroke or spinal cord injury, skeletal immobilization due to trauma, prolonged bed rest, voluntary inactivity, involuntary inactivity, metabolic stress or nutritional insufficiency, cancer, AIDS, fasting, a thyroid gland disorder, diabetes, benign congenital hypotonia, central core disease, burn injury, chronic obstructive pulmonary disease, liver diseases (examples such as fibrosis, cirrhosis), sepsis, renal failure, congestive heart failure, ageing, space travel or time spent in a zero gravity environment.

The term “cachexia” refers to loss of body mass that cannot be reversed nutritionally, and is generally associated with an underlying disease, such as cancer, COPD, AIDS, heart failure, etc. (Evans W J, Morley J E, Arigles J, et al. Cachexia: a new definition. Clinical Nutrition 2008; 27: 793-799).

The term “cancer cachexia” refers to the definition set forth by Fearon et al.: Definition of cancer cachexia: effect of weight loss, reduced food intake, and systemic inflammation on functional status and prognosis; Am J Clin Nutr 2006; 83: 1345-1350.

The term “muscle anabolic agent” refers to any agent which provides muscle growth, such as any pharmaceutical drug and composition comprising said drug/drugs being known to be able to prevent or reverse muscle weakness and/or atrophy in a patient suffering from such a condition, wherein drugs being approved by a health authority for treating patients suffering from muscle weakness or atrophy are particularly preferred. Examples of such agents are an ActRIIB antibody, an ActRIIA antibody, a soluble ActRIIB decoy mimetic, an anti-myostatin antibody, a myostatin propeptide, a myostatin decoy protein that binds ActRIIB but does not activate it, a beta 2 agonist, a Ghrelin agonist, an IGF-1 protein or a mimetic thereof a selective androgen receptor modulator (SARM), growth hormone (GH) agonists/mimetics or follistatin. For example, muscle anabolic agents are those ActRIIB antibodies disclosed in WO2010125003, in particular the ActRIIB antibody designated as Bimagrumab (INN: international non-proprietary name). Furthermore, muscle anabolic agents are those myostatin antibodies disclosed in U.S. Pat. No. 7,632,499 as well as U.S. Pat. No. 8,063,188 and those soluble ActRIIB decoy mimetics disclosed in WO2006012627 and those IGF-1 mimetics disclosed in WO2007146689, WO200040613, WO05033134, WO2006074390, WO2005033134 or WO200040613.

The term “therapeutically effective amount” in the context of administering an therapeutically effective amount as used herein typically refers to an amount of an active ingredient (e.g. Bimagrumab) which, when administered to a subject, is sufficient to provide a therapeutic benefit, e.g. is sufficient for treating the muscle atrophy.

The muscle anabolic agent may be dosed to a subject in any way suitable to provide a therapeutically effective amount to the subject. Actual dosage levels of the active agents in the pharmaceutical compositions of the present invention may be varied so as to obtain an amount of the active agent which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A composition of the present invention can be administered by one or more routes of administration using one or more of a variety of methods known in the art. As will be appreciated by the skilled artisan, the route and/or mode of administration will vary depending upon the desired results. Routes of administration may include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. Alternatively, a composition can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g. Sustained and Controlled Release Drug Delivery Systems, J. R. Robinson, ed., Marcel Dekker, Inc., New York, 1978.

A composition for enteral or parenteral administration is, for example, a unit dosage form, such as a sugar-coated tablet, a tablet, a capsule, a suppository or an ampoule.

The units content of active ingredients in an individual dose need not in itself constitute a therapeutically effective amount, since such an amount can be reached by the administration of a plurality of dosage units. A composition according to the invention may contain, e.g., from about 10% to about 100%, preferably from about 20% to about 60%, of the active ingredients.

If not indicated otherwise, a pharmaceutical composition according to the invention is prepared in a manner known per se, e.g. by means of conventional mixing, granulating, sugar-coating, dissolving or lyophilizing processes. In preparing a composition for an oral dosage form, any of the usual pharmaceutical media may be employed, for example water, glycols, oils, alcohols, carriers, such as starches, sugars, or microcrystalline cellulose, diluents, granulating agents, lubricants, binders, disintegrating agents and the like. Because of their ease of administration, tablets and capsules represent the most advantageous oral dosage unit forms, in which case solid pharmaceutical carriers are obviously employed.

### SEQUENCES

The sequences referred to in the present description are found in Table 1.

TABLE 1
Sequence listing.

| SEQ ID | Accession | UniProt | |
|---|---|---|---|
| NUMBER | number | number | Sequence |
| SEQ ID | IPI00384401 | Q9UL85 | EIVMTQSPATLSVSPGERA |
| NO: 1 | | | TLSCWASQSISSNLAWYQQ |
| | | | KPGQAPRLLIYGASTRATG |
| | | | IPARFSGSGSGTEFTLTIS |
| | | | SLQSEDFAIYHCQQYNSWP |
| | | | PLTFGGGTKVEIKR |
| SEQ ID | IPI00011229 | P07339 | MQPSSLLPLALCLLAAPAS |
| NO: 2 | | | ALVRIPLHKFTSIRRTMSE |
| | | | VGGSVEDLIAKGPVSKYSQ |
| | | | AVPAVTEGPIPEVLKNYMD |
| | | | AQYYGEIGIGTPPQCFTVV |
| | | | FDTGSSNLWVPSIHCKLLD |
| | | | IACWIHHKYNSDKSSTYVK |
| | | | NGTSFDIHYGSGSLSGYLS |
| | | | QDTVSVPCQSASSASALGG |
| | | | VKVERQVFGEATKQPGITF |
| | | | IAAKFDGILGMAYPRISVN |
| | | | NVLPVFDNLMQQKLVDQNI |
| | | | FSFYLSRDPDAQPGGELML |
| | | | GGTDSKYYKGSLSYLNVTR |
| | | | KAYWQVHLDQVEVASGLTL |
| | | | CKEGCEAIVDTGTSLMVGP |
| | | | VDEVRELQKAIGAVPLIQG |
| | | | EYMIPCEKVSTLPAITLKL |
| | | | GGKGYKLSPEDYTLKVSQA |
| | | | GKTLCLSGFMGMDIPPPSG |
| | | | PLWILGDVFIGRYYTVFDR |
| | | | DNNRVGFAEAARL |
| SEQ ID | IPI00940952 | Q0KKI6 | MDMRVPAQLLGLLLLWLRG |
| NO: 3 | | | ARCDIQMTQSPSSLSASVG |
| | | | DRVTITCRASQGIRNDLGW |
| | | | YQQKPGKAPKLLIYDASSL |
| | | | ESGVPSRFSGSGSGTDFTL |
| | | | TISSLQPEDFATYYCQQFN |
| | | | NYPHTFGQGTKVEIKRTVA |
| | | | APSVFIFPPSDEQLKSGTA |
| | | | SVVCLLNNFYPREAKVQWK |
| | | | VDNALQSGNSQESVTEQDS |
| | | | KDSTYSLSSTLTLSKADYE |
| | | | KHKVYACEVTHQGLSSPVT |
| | | | KSFNRGEC |
| SEQ ID | IPI00295741 | P07858 | MWQLWASLCCLLVLANARS |
| NO: 4 | | | RPSFHPLSDELVNYVNKRN |
| | | | TTWQAGHNFYNVDMSYLKR |
| | | | LCGTFLGGPKPPQRVMFTE |
| | | | DLKLPASFDAREQWPQCPT |
| | | | IKEIRDQGSCGSCWAFGAV |
| | | | EAISDRICIHTNAHVSVEV |
| | | | SAEDLLTCCGSMCGDGCNG |
| | | | GYPAEAWNFWTRKGLVSGG |
| | | | LYESHVGCRPYSIPPCEHH |
| | | | VNGSRPPCTGEGDTPKCSK |
| | | | ICEPGYSPTYKQDKHYGYN |
| | | | SYSVSNSEKDIMAEIYKNG |
| | | | PVEGAFSVYSDFLLYKSGV |
| | | | YQHVTGEMMGGHAIRILGW |
| | | | GVENGTPYWLVANSWNTDW |
| | | | GDNGFFKILRGQDHCGIES |
| | | | EVVAGIPRTDQYWEKI |
| SEQ ID | IPI00829956 | A0N5G1 | DIQMTQSPSTLSASVGDRV |
| NO: 5 | | | TITCRASQSISSWLAWYQQ |
| | | | KPGKAPKLLIYKASSLESG |
| | | | VPSRFSGSGSGTEFTLTIS |
| | | | SLQPDDFATYYCQQYNSIE |
| | | | GTFGQGTKVEIKRTVAAPS |
| | | | VF |
| SEQ ID | IPI00297487 | P09668 | MWATLPLLCAGAWLLGVPV |
| NO: 6 | | | CGAAELCVNSLEKFHFKSW |
| | | | MSKHRKTYSTEEYHHRLQT |
| | | | FASNWRKINAHNNGNHTFK |
| | | | MALNQFSDMSFAEIKHKYL |
| | | | WSEPQNCSATKSNYLRGTG |
| | | | PYPPSVDWRKKGNFVSPVK |
| | | | NQGACGSCWTFSTTGALES |
| | | | AIAIATGKMLSLAEQQLVD |
| | | | CAQDFNNHGCQGGLPSQAF |
| | | | EYILYNKGIMGEDTYPYQG |
| | | | KDGYCKFQPGKAIGFVKDV |
| | | | ANITIYDEEAMVEAVALYN |
| | | | PVSFAFEVTQDFMMYRTGI |
| | | | YSSTSCHKTPDKVNHAVLA |
| | | | VGYGEKNGIPYWIVKNSWG |
| | | | PQWGMNGYFLIERGKNMCG |
| | | | LAACASYPIPLV |
| SEQ ID | IPI00478181 | B4DGG2 | MAARPSRATGPRGGQRSRV |
| NO: 7 | | | KPPPGRRLKEQLPPLAAAR |
| | | | AVFAAATAVIMLTEASLSI |
| | | | WGWGSLGIVLFLITFGPFV |
| | | | IFYLTFYILCYVGGGLVVT |
| | | | LLFGKTNSEKYLEQCEHSF |
| | | | LPPTSPGVPKCLEEMKREA |
| | | | RIIKIDRRLTGANIIDEPL |
| | | | QQVIQFSLRDYVQYWYYTL |
| | | | SDDESFLLEIRQTLQNALI |
| | | | QFATRSKEIDWQPYFTTRI |
| | | | VDDFGTHLRVFRKAQQKIT |
| | | | EKDDQVKGTAEDLVDTFFE |
| | | | VEVEMEKEVCRDLVCTSPK |
| | | | DEEGFLRDLCEVLLYLLLP |
| | | | PGDFQNKIMRYFVREILAR |
| | | | GILLPLINQLSDPDYINQY |
| | | | VIWMIRDSNCNYEAFMNII |
| | | | KLSDNIGELEAVRDKAAEE |
| | | | LQYLRSLDTAGDDINTIKN |
| | | | QINSLLFVKKVCDSRIQRL |
| | | | QSGKEINTVKLAANFGKLC |
| | | | TVPLDSILVDNVALQFFMD |
| | | | YMQQTGGQAHLFFWMTVEG |
| | | | YRVTAQQQLEVLLSRQRDG |
| | | | KHQTNQTKGLLRAAAVGIY |
| | | | EQYLSEKASPRVTVDDYLV |
| | | | AKLADTLNHEDPTPEIFDD |
| | | | IQRKVYELMLRDERFYPSF |
| | | | RQNALYVRMLAELDMLKDP |
| | | | SFRGSDDGDGESFNGSPTG |
| | | | SINLSLDDLSNVSSDDSVQ |
| | | | LHAYISDTGVCNDHGKTYA |
| | | | LYAITVHRRNLNSEEMWKT |
| | | | YRRYSDFHDFHMRITEQFE |
| | | | SLSSILKLPGKKTFNNMDR |
| | | | DFLEKRKKDLNAYLQLLLA |
| | | | PEMMKASPALAHYVYDFLE |
| | | | NKAYSKGKGDFARKMDTFV |
| | | | NPLRNSMRNVSNAVKSLPD |
| | | | SLAEGMTKMSDNMGKMSER |
| | | | LGQDIKQSFFKVPPLIPKT |
| | | | DSDPEHRRVSAQLDDNVDD |
| | | | NIPLRVMLLLMDEVFDLKE |
| | | | RNQWLRRNIKNLLQQLIRA |
| | | | TYGDTINRKIVDHVDWMTS |
| | | | PEQVADSVKRFRDAFWPNG |
| | | | ILAEAVPCRDKSIRMRTRV |
| | | | AGKTKLLAIMPDELKHIIG |
| | | | AETTRKGILRVFEMFQHNQ |
| | | | LNRRMVYVFLEGFLETLFP |
| | | | QYKFRELFNKLHSRSKQMQ |
| | | | KYKQKLQTTQAPSLQKR |
| SEQ ID | IPI00646773 | P06396-2 | MVVEHPEFLKAGKEPGLQI |
| NO: 8 | | | WRVEKFDLVPVPTNLYGDF |
| | | | FTGDAYVILKTVQLRNGNL |
| | | | QYDLHYWLGNECSQDESGA |
| | | | AAIFTVQLDDYLNGRAVQH |
| | | | REVQGFESATFLGYFKSGL |
| | | | KYKKGGVASGFKHVVPNEV |
| | | | VVQRLFQVKGRRVVRATEV |
| | | | PVSWESFNNGDCFILDLGN |
| | | | NIHQWCGSNSNRYERLKAT |
| | | | QVSKGIRDNERSGRARVHV |
| | | | SEEGTEPEAMLQVLGPKPA |
| | | | LPAGTEDTAKEDAANRKLA |
| | | | KLYKVSNGAGTMSVSLVAD |
| | | | ENPFAQGALKSEDCFILDH |
| | | | GKDGKIFVWKGKQANTEER |
| | | | KAALKTASDFITKMDYPKQ |
| | | | TQVSVLPEGGETPLFKQFF |
| | | | KNWRDPDQTDGLGLSYLSS |
| | | | HIANVERVPFDAATLHTST |
| | | | AMAAQHGMDDDGTGQKQIW |
| | | | RIEGSNKVPVDPATYGQFY |
| | | | GGDSYIILYNYRHGGRQGQ |
| | | | IIYNWQGAQSTQDEVAASA |
| | | | ILTAQLDEELGGTPVQSRV |
| | | | VQGKEPAHLMSLFGGKPMI |
| | | | IYKGGTSREGGQTAPASTR |
| | | | LFQVRANSAGATRAVEVLP |
| | | | KAGALNSNDAFVLKTPSAA |
| | | | YLWVGTGASEAEKTGAQEL |
| | | | LRVLRAQPVQVAEGSEPDG |
| | | | FWEALGGKAAYRTSPRLKD |
| | | | KKMDAHPPRLFACSNKIGR |
| | | | FVIEEVPGELMQEDLATDD |
| | | | VMLLDTWDQVFVWVGKDSQ |
| | | | EEEKTEALTSAKRYIETDP |
| | | | ANRDRRTPITVVKQGFEPP |
| | | | SFVGWFLGWDDDYWSVDPL |
| | | | DRAMAELAA |
| SEQ ID | IPI00387100 | P01608 | DIQMTQSPSSLSASVGDRV |
| NO: 9 | | | TITCQASQDISIFLNWYQQ |
| | | | KPGKAPKLLIYDASKLEAG |
| | | | VPSRFSGTGSGTDFTFTIS |
| | | | SLQPEDIATYYCQQFDNLP |
| | | | LTFGGGTKVDFKR |
| SEQ ID | IPI00026314 | P06396-1 | MAPHRPAPALLCALSLALC |
| NO: 10 | | | ALSLPVRAATASRGASQAG |
| | | | APQGRVPEARPNSMVVEHP |
| | | | EFLKAGKEPGLQIWRVEKF |
| | | | DLVPVPTNLYGDFFTGDAY |
| | | | VILKTVQLRNGNLQYDLHY |
| | | | WLGNECSQDESGAAAIFTV |
| | | | QLDDYLNGRAVQHREVQGF |
| | | | ESATFLGYFKSGLKYKKGG |
| | | | VASGFKHVVPNEVVVQRLF |
| | | | QVKGRRVVRATEVPVSWES |
| | | | FNNGDCFILDLGNNIHQWC |
| | | | GSNSNRYERLKATQVSKGI |
| | | | RDNERSGRARVHVSEEGTE |
| | | | PEAMLQVLGPKPALPAGTE |
| | | | DTAKEDAANRKLAKLYKVS |
| | | | NGAGTMSVSLVADENPFAQ |
| | | | GALKSEDCFILDHGKDGKI |
| | | | FVWKGKQANTEERKAALKT |
| | | | ASDFITKMDYPKQTQVSVL |
| | | | PEGGETPLFKQFFKNWRDP |
| | | | DQTDGLGLSYLSSHIANVE |
| | | | RVPFDAATLHTSTAMAAQH |
| | | | GMDDDGTGQKQIWRIEGSN |
| | | | KVPVDPATYGQFYGGDSYI |
| | | | ILYNYRHGGRQGQIIYNWQ |
| | | | GAQSTQDEVAASAILTAQL |
| | | | DEELGGTPVQSRVVQGKEP |
| | | | AHLMSLFGGKPMIIYKGGT |
| | | | SREGGQTAPASTRLFQVRA |
| | | | NSAGATRAVEVLPKAGALN |
| | | | SNDAFVLKTPSAAYLWVGT |
| | | | GASEAEKTGAQELLRVLRA |
| | | | QPVQVAEGSEPDGFWEALG |
| | | | GKAAYRTSPRLKDKKMDAH |
| | | | PPRLFACSNKIGRFVIEEV |
| | | | PGELMQEDLATDDVMLLDT |
| | | | WDQVFVWVGKDSQEEEKTE |
| | | | ALTSAKRYIETDPANRDRR |
| | | | TPITVVKQGFEPPSFVGWF |
| | | | LGWDDDYWSVDPLDRAMAE |
| | | | LAA |
| SEQ ID | IPI00885174 | Q96PX9 | MEALRNPMPLGSSEEALGD |
| NO: 11 | | | LACSSLTGASRDLGTGAVA |
| | | | SGTQEETSGPRGDPQQTPS |
| | | | LEKERHTPSRTGPGAAGRT |
| | | | LPRRSRSWERAPRSSRGAQ |
| | | | AAACHTSHHSAGSRPGGHL |
| | | | GGQAVGTPNCVPVEGPGCT |
| | | | KEEDVLASSACVSTDGGSL |
| | | | HCHNPSGPSDVPARQPHPE |
| | | | QEGWPPGTGDFPSQVPKQV |
| | | | LDVSQELLQSGVVTLPGTR |
| | | | DRHGRAVVQVRTRSLLWTR |
| | | | EHSSCAELTRLLLYFHSIP |
| | | | RKEVRDLGLVVLVDARRSP |
| | | | AAPAVSQALSGLQNNTSPI |
| | | | IHSILLLVDKESAFRPDKD |
| | | | AIIQCEVVSSLKAVHKFVD |
| | | | SCQLTADLDGSFPYSHGDW |
| | | | ICFRQRLEHFAANCEEAII |
| | | | FLQNSFCSLNTHRTPRTAQ |
| | | | EVAELIDQHETMMKLVLED |
| | | | PLLVSLRLEGGTVLARLRR |
| | | | EELGTEDSRDTLEAATSLY |
| | | | DRVDEEVHRLVLTSNNRLQ |
| | | | QLEHLRELASLLEGNDQQS |
| | | | CQKGLQLAKENPQRTEEMV |
| | | | QDFRRGLSAVVSQAECREG |
| | | | ELARWTRSSELCETVSSWM |
| | | | GPLDPEACPSSPVAECLRS |
| | | | CHQEATSVAAEAFPGAGVA |
| | | | VLKPHALGKPWASQQDLWL |
| | | | QYPQTRLRLEEALSEAAPD |
| | | | PSLPPLAQSPPKHERAQEA |
| | | | MRRHQKPPSFPSTDSGGGA |
| | | | WEPAQPLSGLPGRALLCGQ |
| | | | DGEPLGPGLCALWDPLSLL |
| | | | RGLPGAGATTAHLEDSSAC |
| | | | SSEPTQTLASRPRKHPQKK |
| | | | MIKKTQSFEIPQPDSGPRD |
| | | | SCQPDHTSVFSKGLEVTST |
| | | | VATEKKLPLWQHARSPPVT |
| | | | QSRSLSSPSGLHPAEEDGR |
| | | | QQVGSSRLRHIMAEMIATE |
| | | | REYIRCLGYVIDNYFPEME |
| | | | RMDLPQGLRGKHHVIFGNL |
| | | | EKLHDFHQQHFLRELERCQ |
| | | | HCPLAVGRSFLRHEEQFGM |
| | | | YVIYSKNKPQSDALLSSHG |
| | | | NAFFKDKQRELGDKMDLAS |
| | | | YLLRPVQRVAKYALLLQDL |
| | | | LKEASCGLAQGQELGELRA |
| | | | AEVVVCFQLRHGNDLLAMD |
| | | | AIRGCDVNLKEQGQLRCRD |
| | | | EFIVCCGRKKYLRHVFLFE |
| | | | DLILFSKTQKVEGSHDVYL |
| | | | YKQSFKTAEIGMTENVGDS |
| | | | GLRFEIWFRRRQKSQDTYI |
| | | | LQASSAEVKSAWTDVIGRI |
| | | | LWRQALKSRELRIQEMASM |
| | | | GIGNQPFMDVKPRDRTPDC |
| | | | AVISDGAPKCAVMSDRVPD |
| | | | SIVKGTESQMRGSTAVSSS |
| | | | DHAAPFKRPHSTISDSSTS |
| | | | SSSSQSSSILGSLGLLVSS |
| | | | SPAHPGLWSPAHSPWSSDI |
| | | | RACVEEDEPEPELETGTQA |
| | | | AVCEGAPAVLLSRTRQA |
| SEQ ID | IPI00398221 | A4D2G3 | MTKNPKVNAIKTKALDALG |
| NO: 12 | | | HQYEEAASFQYMRTQSEHQ |
| | | | SPTSLLLNRSDGEKHIKTC |
| | | | YEMGGNQTSITEFLLLGFP |
| | | | IGPRIQMLLFGLFSLFYIF |
| | | | ILLGNGTILGLISLDSRLH |
| | | | TPMYFFLSHLAVVDIACAC |
| | | | STVPQMLVNLLHPAKPISF |
| | | | AGCMTQMFLFLSFAHTECL |
| | | | LLVVMSYDRYVAICHPLRY |
| | | | STIMTWKVCITLALTSWIL |
| | | | GVLLALVHLVLLLPLSFCG |
| | | | PQKLNHFFCEIMAVLKLAC |
| | | | ADTHINEVMVLAGAVSVLV |
| | | | GAFFSTVISYVHILCAILK |
| | | | IQSGEGCQKAFSICSSHLC |
| | | | VVGLFYGTAIIMYVEPQYE |
| | | | SPKEQKKYLLLFHSLFNPM |
| | | | LNPLIYSLRNKEVQENSEV |
| | | | QNKVLNFVMFTVQNQCLIL |
| | | | VVMSYDRYVAICHPFQYTV |
| | | | IMSWRVCTILVLTSWSCGF |
| | | | ALSLVHEILLLRLPFCGPR |
| | | | DVNHLFCEILSVLKLACAD |
| | | | TWVNQVVIFATCVFVLVGP |
| | | | LSLILVSYMHILGAILKIQ |
| | | | TKEGRIKAFSTCSSHLCVV |
| | | | GLFFGIAMVVYMVPDSNQR |
| | | | EEQEKMLSLFHSVFNPMLN |
| | | | PLIYSLRNAQLKGALHRAL |
| | | | QRKRSMRTVYGLCL |
| SEQ ID | IPI00004067 | Q93073-1 | MDRAPTEQNVKLSAEVEPF |
| NO: 13 | | | IPQKKSPDTFMIPMALPND |
| | | | NGSVSGVEPTPIPSYLITC |
| | | | YPFVQENQSNRQFPLYNND |
| | | | IRWQQPNPNPTGPYFAYPI |
| | | | ISAQPPVSTEYTYYQLMPA |
| | | | PCAQVMGFYHPFPTPYSNT |
| | | | FQAANTVNAITTECTERPS |
| | | | QLGQVFPLSSHRSRNSNRG |
| | | | SVVPKQQLLQQHIKSKRPL |
| | | | VKNVATQKETNAAGPDSRS |
| | | | KIVLLVDASQQTDFPSDIA |
| | | | NKSLSETTATMLWKSKGRR |
| | | | RRASHPTAESSSEQGASEA |
| | | | DIDSDSGYCSPKHSNNQPA |
| | | | AGALRNPDSGTMNHVESSM |
| | | | CAGGVNWSNVTCQATQKKP |
| | | | WMEKNQTFSRGGRQTEQRN |
| | | | NSQVGFRCRGHSTSSERRQ |
| | | | NLQKRPDNKHLSSSQSHRS |
| | | | DPNSESLYFEDEDGFQELN |
| | | | ENGNAKDENIQQKLSSKVL |
| | | | DDLPENSPINIVQTPIPIT |
| | | | TSVPKRAKSQKKKALAAAL |
| | | | ATAQEYSEISMEQKKLQEA |
| | | | LSKAAGKKNKTPVQLDLGD |
| | | | MLAALEKQQQAMKARQITN |
| | | | TRPLSYTVVTAASFHTKDS |
| | | | TNRKPLTKSQPCLTSFNSV |
| | | | DIASSKAKKGKEKEIAKLK |
| | | | RPTALKKVILKEREEKKGR |
| | | | LTVDHNLLGSEEPTEMHLD |
| | | | FIDDLPQEIVSQEDTGLSM |
| | | | PSDTSLSPASQNSPYCMTP |
| | | | VSQGSPASSGIGSPMASST |
| | | | ITKIHSKRFREYCNQVLCK |
| | | | EIDECVTLLLQELVSFQER |
| | | | IYQKDPVRAKARRRLVMGL |
| | | | REVTKHMKLNKIKCVIISP |
| | | | NCEKIQSKGGLDEALYNVI |
| | | | AMAREQEIPFVFALGRKAL |
| | | | GRCVNKLVPVSVVGIFNYF |
| | | | GAESLFNKLVELTEEARKA |
| | | | YKDMVAAMEQEQAEEALKN |
| | | | VKKVPHHMGHSRNPSAASA |
| | | | ISFCSVISEPISEVNEKEY |
| | | | ETNWRNMVETSDGLEASEN |
| | | | EKEVSCKHSTSEKPSKLPF |
| | | | DTPPIGKQPSLVATGSTTS |
| | | | ATSAGKSTASDKEEVKPDD |
| | | | LEWASQQSTETGSLDGSCR |
| | | | DLLNSSITSTTSTLVPGML |
| | | | EEEEDEDEEEEEDYTHEPI |
| | | | SVEVQLNSRIESWVSETQR |
| | | | TMETLQLGKTLNGSEEDNV |
| | | | EQSGEEEAEAPEVLEPGMD |
| | | | SEAWTADQQASPGQQKSSN |
| | | | CSSLNKEHSDSNYTTQTT |
| SEQ ID | IPI00387113 | P01619 | ZIVLTZSPGTLSLSPGZRA |
| NO: 14 | | | ALSCRASQSLSGNYLAWYQ |
| | | | QKPGQAPRLLMYGVSSRAT |
| | | | GIPDRFSGSGSGADFTLTI |
| | | | SRLZPEDFAVYYCQQYGSS |
| | | | PFTFGQGSKLEIK |
| SEQ ID | IPI00816799 | A0N5G5 | EIVLTQSPATLSLSPGERA |
| NO: 15 | | | SLSCRASQSVATYLAWYQH |
| | | | KPGQAPRLLIYDASNRATG |
| | | | IPARFSGSGSGTDFTLTIS |
| | | | SLEPADFAVYYCQHRNNWP |
| | | | PLFTFGPGTRVDVKRTVAA |
| | | | PSVF |
| SEQ ID | IPI00552510 | B1AL98 | XFKMQDYNKDDMSYRRISA |
| NO: 16 | | | VEPKTALPFNRFLPNKSRQ |
| | | | PSYVPAPLRKKKPDKHEDN |
| | | | RRSWASPVYTEADGTFSSQ |
| | | | FLLLQALQTYSDDILSSET |
| | | | HTKIDPTSGPRLITRRKNL |
| | | | SYAPGYRRDDLEMAALDPD |
| | | | LENDDFFVRKTGAFHANPY |
| | | | VLRAFEDFRKFSEQDDSVE |
| | | | RDIILQCREGELVLPDLEK |
| | | | DDMIVRRIPAQKKEVPLSG |
| | | | APDRYHPVPFPEPWTLPPE |
| | | | IQAKFLCVLERTCPSKEKS |
| | | | NSCRILVPSYRQKKDDMLT |
| | | | RKIQSWKLGTTVPPISFTP |
| | | | GPCSEADLKRWEAIREASR |
| | | | LRHKKRLMVERLFQKIYGE |
| | | | NGSKSMSDVSAEDVQNLRQ |
| | | | LRYEEMQKIKSQLKEQDQK |
| | | | WQDDLAKWKDRRKSYTSDL |
| | | | QKKKEEREEIEKQALEKSK |
| | | | RSSKTFKEMLQDRESQNQK |
| | | | STVPSRRRMYSFDDVLEEG |
| | | | KRPPTMTVSEASYQSERVE |
| | | | EKGATYPSEIPKEDSTTFA |
| | | | KREDRVTTEIQLPSQSPVE |
| | | | EQSPASLSSLRSRSTQMES |
| | | | TRVSASLPRSYRKTDTVRL |
| | | | TSVVTPRPFGSQTRGISSL |
| | | | PRSYTMDDAWKYNGDVEDI |
| | | | KRTPNNVVSTPAPSPDASQ |
| | | | LASSLSSQKEVAATEEDVT |
| | | | RLPSPTSPFSSLSQDQAAT |
| | | | SKATLSSTSGLDLMSESGE |
| | | | GEISPQREVSRSQDQFSDM |
| | | | RISINQTPGKSLDFGFTIK |
| | | | WDIPGIFVASVEAGSPAEF |
| | | | SQLQVDDEIIAINNTKFSY |
| | | | NDSKEWEEAMAKAQETGHL |
| | | | VMDVRRYGKAGSPETKWID |
| | | | ATSGIYNSEKSSNLSVTTD |
| | | | FSESLQSSNIESKEINGIH |
| | | | DESNAFESKASESISLKNL |
| | | | KRRSQFFEQGSSDSVVPDL |
| | | | PVPTISAPSRWVWDQEEER |
| | | | KRQERWQKEQDRLLQEKYQ |
| | | | REQEKLREEWQRAKQEAER |
| | | | ENSKYLDEELMVLSSNSMS |
| | | | LTTREPSLATWEATWSEGS |
| | | | KSSDREGTRAGEEERRQPQ |
| | | | EEVVHEDQGKKPQDQLVIE |
| | | | RERKWEQQLQEEQEQKRLQ |
| | | | AEAEEQKRPAEEQKRQAEI |
| | | | ERETSVRIYQYRRPVDSYD |
| | | | IPKTEEASSGFLPGDRNKS |
| | | | RSTTELDDYST |

### Examples

The following examples are given for the purpose of illustrating various embodiments of the invention and are not meant to limit the present invention in any fashion.

### 1. Sampling

Urine samples from 63 individuals were obtained. These included 23 samples from cancer patients who had weight loss of ≧10 of premorbid body weight (8 gastric/EGJ/esophageal cancer, 13 pancreatic cancer, 1 duodenum cancer and 1 small bowel cancer); 32 samples from cancer patients who had weight loss of <10% (22 gastric/EGJ/esophageal cancer, 9 pancreatic cancer and 1 pancreas/duodenum cancer); and 8 from healthy volunteers. Premorbid body weight refers to the patient's known weight before the diagnosis of cancer, obtained either from the medical history or from medical records. In addition, 5 normal control samples were used. The samples were analyzed in a blinded fashion at Novartis, Cambridge.

### 2. Biomarker Assessments

20 μL of urine was used for protein concentration measurement. The Bradford assay, well known to a person skilled in the art, was performed to measure the total protein concentration for each sample. Urine samples were normalized by the total protein amount. Various volume of urine was taken from each sample (ranging from 70 μl to 1.95 ml). 50 mM Tris buffer pH 7.5 was added to top each sample to the same final volume. Protease inhibitor was added to each sample to minimize proteolysis during the sample preparation. Five μg of enolase was added to each sample as an internal standard. All urine samples were filtered through a 3 kDa MWCO filter. The high MW content proteins were used for this study. The urine protein samples were denatured (urea), reduced (DTT) and alkylated (iodoacetamide), before trypsin was added to each sample for an overnight digestion at 37° C.

At the end of the digestion, formic acid (final concentration 2%) was added to each sample to stop the digestion. A MCX plate was used to clean the digested sample. Aliquots of each cleaned, reconstituted sample was injected to LC-MS/MS for analysis. LC separation was achieved on a 300 um×150 mm C18 column, using a 90 min gradient from 5% B (acetonitrile in 0.1% formic acid) to 45% B with a flow rate of 15 μL/min. Mobile phase A was water with 0.1% formic acid. The eluted peptides were measured by the LTQ Orbitrap Velos mass spectrometer. The top 20 most abundant peptide peaks were selected every few seconds for fragmentation in the MS and fragment spectrum of each selected peptide was recorded and used for sequencing and identification. The peptide peak intensity at the MS level, after normalization, reflected the abundance of the corresponding peptide, and used in the quantitative analysis. Three LC-MS/MS runs were performed for each patient sample.

### 3. Data Analysis

The primary analysis was of the correlation of urine proteins or peptides with weight loss reported by the patient up to the time of urine collection.

All acquired raw data files were imported into Progenesis software, well known to a person skilled in the art. The comprehensive alignment involved the use of one run as the reference, automatic placement of landmarks (vectors), calculation of non-linear mapping between retention times of reference runs and those being aligned. Peak picking was performed for each run and features from all runs were aggregated to a master table, and subsequently, features with m/z values and retention time were mapped to all runs, and peak areas were extracted for each feature in each run. For protein identification, MS/MS data associated with each feature was exported to Mascot for protein sequence database search. Mascot search results were imported back to Progenesis for feature annotation. Finally, the data table with peptide and protein sequences, intensities, Mascot scores etc. were exported as CSV files for statistical analysis.

Data tables with intensities of peptides and proteins were exported in text format and imported into the R statistical software package for analysis. Multivariate analysis including PCA, hierarchical clustering, and random forest regression to assess the overall quality and structure of the data was performed.

Reproducibility of the technical repeat measurements was good, and no obvious outlier spectra were observed. Sex and age also did not appear as important factors in the data set.

Univariate regression between patient weight loss and protein intensities resulted in a large number of significant correlations (11 proteins with p-values <10⁻⁶, not adjusted for multiple testing). All of these had a negative correlation with weight loss, i.e. higher intensity in cachectic patients.

A demographic summary of the sample population is found in Table 2.

TABLE 2
Demographic summary.

| | | Novartis |
|---|---|---|
| | | Samples |
| | | N = 68 |
| Age (years) | Mean | 62.9 |
| | SD | 9.5 |
| | Median | 64.0 |
| | Range | 42-83 |
| Age group - n (%) | 18-64 years | 31 (46) |
| | 65-84 years | 32 (54) |
| | ≧85 years | 0 |
| Gender - n (%) | Male | 41 (60) |
| | Female | 22 (40) |
| Race - n (%) | Caucasian | 63 (100) |
| | Black | 0 |
| | Oriental | 0 |
| | Native | 0 |
| | American | |
| | Other | 0 |
| BMI (kg/m2) | Mean | 25.85 |
| | SD | 4.35 |
| | Median | 25.60 |
| | Range | 17.9-35.9 |

BMI = body mass index

An overview of disease characteristics per treatment group is found in Table 3.

TABLE 3
Disease characteristics per treatment group.

| | | Novartis |
|---|---|---|
| | | Samples |
| | | N = 68 |
| Tumor Type - n (%) | Esophageal | 16 (24) |
| | EGJ | 11 (16) |
| | Gastric | 9 (13) |
| | Pancreatic | 22 (32) |
| | Duodenum | 1 (1) |
| | Small bowel | 1 (1) |
| | Control | 8 (12) |
| Weight Loss - %* | Mean | −8.4% |
| | SD | 9.8% |
| | Median | −6.1% |
| | Range | 0-43% |
| | <10%, n | 40 |
| | >10%, n | 23 |

*does not include healthy controls

### 4. Results

Univariate regression between patient weight loss and protein intensities resulted in a large number of significant correlations (11 proteins with p-values <10⁻⁶, not adjusted for multiple testing).

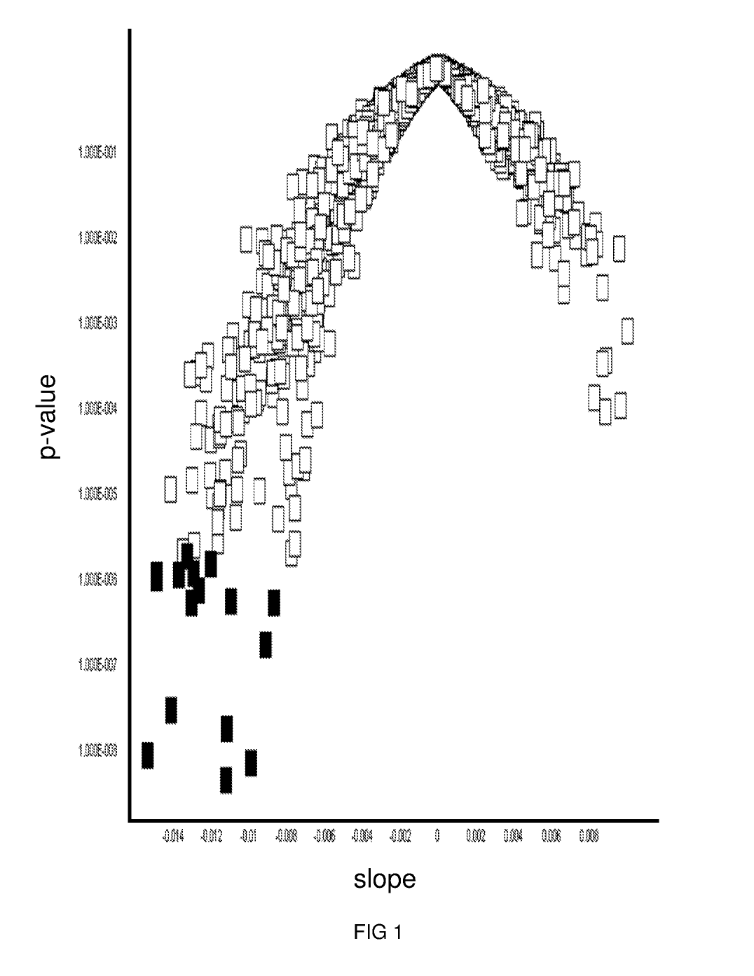

Statistical analysis of urine protein intensity vs. previous weight loss by ANOVA has identified proteins with statistically significant correlations, as shown in FIG. 1. The x-axis shows the slope of the association of the urine concentration of a protein vs. weight loss in the patient from whom the urine sample was obtained. The y-axis indicates the nominal p-value of the regression line slope. Slope in this analysis corresponds to the magnitude of effect, with higher (positive or negative) values indicating a bigger change in protein intensity between patients' samples. The plot is skewed to the left, showing that most proteins with a good correlation with weight loss are negatively correlated, that is having higher intensities in patients with higher weight loss. The most significantly changed proteins in this plot are characterized by low p-values and high (negative) slope, corresponding to points in the lower left corner.

This analysis indicates that a robust range of proteins was identified, and that a relatively small group was associated with previous weight loss.

The top 16 polypeptides, all had nominal p-values less than 2×10⁻⁶. These top 16 polypeptides are the biomarkers that are indicative of cancer cachexia, and they are summarized in Table 4, and their sequences are found in Table 1.

TABLE 4
List of most significant proteins.

| | | slope wt. | |
|---|---|---|---|
| Accession | Description | loss | p value |
| IPI00384401 | Myosin-reactive immunoglobulin | −0.0112 | 4.44E−09 |
| | kappa chain variable region | | |
| | (Fragment) | | |
| IPI00011229 | CTSD Cathepsin D | −0.00992 | 6.90E−09 |
| IPI00940952 | IGKC Ig kappa chain C region | −0.01539 | 8.80E−09 |
| IPI00295741 | CTSB Cathepsin B | −0.0112 | 1.74E−08 |
| IPI00829956 | Rheumatoid factor C6 light | −0.0142 | 2.97E−08 |
| | chain (Fragment) | | |
| IPI00297487 | CTSH Cathepsin H | −0.00916 | 1.69E−07 |
| IPI00478181 | SNX13 cDNA FLJ56070, highly | −0.00872 | 5.25E−07 |
| | similar to Sorting nexin-13 | | |
| IPI00646773 | GSN Isoform 2 of Gelsolin | −0.01309 | 5.25E−07 |
| IPI00387100 | Ig kappa chain V-I region Roy | −0.0109 | 5.50E−07 |
| IPI00026314 | GSN Isoform 1 of Gelsolin | −0.01268 | 7.49E−07 |
| IPI00885174 | PLEKHG4B Pleckstrin homology | −0.01491 | 1.02E−06 |
| | domain-containing family G | | |
| | member 4B | | |
| IPI00398221 | OR2A25 Olfactory receptor 2A25 | −0.01374 | 1.12E−06 |
| IPI00004067 | SECISBP2L Isoform 1 of | −0.01492 | 1.12E−06 |
| | Selenocysteine insertion | | |
| | sequence-binding protein 2- | | |
| IPI00387113 | Ig kappa chain V-III region | −0.0129 | 1.15E−06 |
| | B6 | | |
| IPI00816799 | Rheumatoid factor D5 light | −0.0120 | 1.50E−06 |
| IPI00552510 | chain (Fragment) LMO7 LIM | −0.01329 | 1.87E−06 |
| | domain 7 | | |

Consequently, the top 16 polypeptides (Tables 1 and 4) are indicative of cancer cachexia. Since cancer cachexia is a muscle atrophy syndrome, it is reasonable to expect that the occurrence of the identified significant polypeptides of Table 4 would also be predictive of other muscle atrophy syndromes, such as disuse atrophy, cachexia, sporadic inclusion body myositis, renal failure, AIDS (acquired immunodeficiency syndrome) and COPD (chronic obstructive pulmonary disease).

Thus, in one embodiment the invention comprises biomarkers indicative of disuse atrophy, cachexia, sporadic inclusion body myositis, renal failure, AIDS (acquired immunodeficiency syndrome) and COPD (chronic obstructive pulmonary disease).

The biomarkers of the present invention may be used singly, or in combination.

In one embodiment, the biomarkers are used in a combination of 2, 3, 4, 5, 6, 7, 8, 9 or 10 of the biomarkers of SEQ ID NOs 1 to 16.

The expression of said one or more biomarkers can be detected using any method known to a person having ordinary skill in the art. In one embodiment, the expression of said one or more biomarkers can be detected using a reagent that detects said one or more biomarkers. Said reagent can be any reagent that specifically detects said one or more biomarkers. Said reagent can be an antibody (natural or synthetic) or a fragment thereof specific for the biomarker, a peptide, a nucleic acid, or any other reagent that can specifically detect a biomarker.

In another embodiment, said reagent is directly or indirectly labeled with a detectable substance. The detectable substance may be, for example, selected, e. g., from a group consisting of radioisotopes, fluorescent compounds, enzymes, and enzyme co-factor. Methods of labeling antibodies are well known in the art.

In yet another embodiment, the expression of said one or more biomarkers can be detected by ELISA, RCA immunoassay, chemiluminescence, thin-film optical biosensor, proton resonance technology, protein microarray assay or any other detection method known in the art.

In another embodiment, the expression of said one or more biomarker is detected using mass spectroscopy, such as LC-MS or LC-MS/MS, well known to a person skilled in the art.

In one embodiment, the biomarkers according to other embodies described herewith are supplemented with additional data, such as CD scan of the patient.

1) A method of selectively treating muscle atrophy in a patient, comprising selectively administering a therapeutically effective amount of a muscle anabolic agent to the patient on the basis of the patient having significantly increased levels of the polypeptides in the group consisting of SEQ ID NOs 1 to 16, in urine.

2) A method of selectively treating a patient having muscle atrophy with a muscle anabolic agent, comprising:
a) selecting the patient for treatment with a muscle anabolic agent on the basis of a the patient having significantly increased levels of the polypeptides in the group consisting of SEQ ID NOs 1 to 16, in urine; and
b) thereafter, administering a therapeutically effective amount of a muscle anabolic agent to the patient.

3) A method of selectively treating a patient having muscle atrophy with a muscle anabolic agent, comprising:
a) assaying a urine sample from the patient for the polypeptides in the group consisting of SEQ ID NOs 1 to 16;
b) thereafter, selecting the patient for treatment with a muscle anabolic agent on the basis of the urine sample from the patient having significantly increased levels of the polypeptides in the group consisting of SEQ ID NOs 1 to 16; and
c) thereafter, administering a therapeutically effective amount of a muscle anabolic agent to the patient.

4) A method of predicting the likelihood that a patient having muscle atrophy will respond to treatment with a muscle anabolic agent, comprising assaying a urine sample from the patient for the presence or absence of the polypeptides in the group consisting of SEQ ID NOs 1 to 16, wherein:
a) significantly increased levels of the polypeptides in the group consisting of SEQ ID NOs 1 to 16 in urine is indicative of an increased likelihood that the patient will respond to treatment with a muscle anabolic agent; and
b) the absence of significantly increased levels of the polypeptides in the group consisting of SEQ ID NOs 1 to 16 in urine is indicative of a decreased likelihood that the patient will respond to treatment with a muscle anabolic agent.

5) A method of producing a transmittable form of information for predicting the responsiveness of a patient having muscle atrophy to treatment with a muscle anabolic agent, comprising:
a) determining an increased likelihood of the patient responding to treatment with a muscle anabolic agent based on significantly increased levels of the polypeptides in the group consisting of SEQ ID NOs 1 to 16, in urine; and
b) recording the result of the determining step on a tangible or intangible media form for use in transmission.

6) A method of predicting the likelihood that a patient having muscle atrophy will respond to treatment with a muscle anabolic agent, comprising:
a) providing a urine sample from the patient;
b) assaying a urine sample from the patient for levels of the polypeptides in the group consisting of SEQ ID NOs 1 to 16 in the sample; and
c) reporting whether the polypeptides in the group consisting of SEQ ID NOs 1 to 16 in urine is present in significantly increased levels in the sample, wherein the significantly increased levels of the polypeptides in the group consisting of SEQ ID NOs 1 to 16 in urine is indicative of an increased likelihood of response to a muscle anabolic agent.

7) A method of selectively predicting development of muscle atrophy in a patient, comprising identifying a patient on the basis of the patient having significantly increased levels of the polypeptides in the group consisting of SEQ ID NOs 1 to 16, in urine, wherein significantly increased levels of the polypeptides in the group consisting of SEQ ID NOs 1 to 16 in urine is indicative of a predisposition for developing muscle atrophy.

8) A method of selectively predicting development of muscle atrophy in a patient, comprising:
a) assaying a urine sample from the patient for the polypeptides in the group consisting of SEQ ID NOs 1 to 16;
b) thereafter, selecting the patient for treatment with a muscle anabolic agent on the basis of the urine sample from the patient having significantly increased levels of the polypeptides in the group consisting of SEQ ID NOs 1 to 16.

9) A method of predicting the likelihood that a patient will develop muscle atrophy, comprising assaying a urine sample from the patient for the presence or absence of the polypeptides in the group consisting of SEQ ID NOs 1 to 16, wherein:
a) significantly increased levels of the polypeptides in the group consisting of SEQ ID NOs 1 to 16 in urine is indicative of an increased likelihood that the patient will develop muscle atrophy; and
b) the absence of significantly increased levels of the polypeptides in the group consisting of SEQ ID NOs 1 to 16 in urine is indicative of a decreased likelihood that the patient will develop muscle atrophy.

10) A method of producing a transmittable form of information for predicting the if a patient will develop muscle atrophy, comprising:
a) determining an increased likelihood of the patient developing muscle atrophy based on significantly increased levels of the polypeptides in the group consisting of SEQ ID NOs 1 to 16, in urine; and
b) recording the result of the determining step on a tangible or intangible media form for use in transmission.

11) A method of predicting the likelihood that a patient will develop muscle atrophy, comprising:
a) providing a urine sample from the patient;
b) assaying a urine sample from the patient for levels of the polypeptides in the group consisting of SEQ ID NOs 1 to 16 in the sample; and
c) reporting whether the polypeptides in the group consisting of SEQ ID NOs 1 to 16 in urine is present in significantly increased levels in the sample, wherein the significantly increased levels of the polypeptides in the group consisting of SEQ ID NOs 1 to 16 in urine is indicative of an increased likelihood of the patient developing muscle atrophy.

12) The method according to claim 3, wherein the step of assaying comprises a technique selected from the group consisting of immunoassays, immunohistochemistry, ELISA, flow cytometry, Western blot, HPLC, mass spectrometry, alone or in combination.

13) The method according to claim 6 or 11, wherein the technique is LC-MS/MS.

14) (canceled)

15) (canceled)

16) (canceled)

17) (canceled)

18) (canceled)

19) (canceled)

20) (canceled)

21) A kit for use in predicting the likelihood that a patient having muscle atrophy will respond to treatment with a muscle anabolic agent comprising,
a) at least one probe capable of detecting the presence of the polypeptides in the group consisting of SEQ ID NOs 1 to 16; and
b) instructions for using the probe to assay a biological sample from the muscle atrophy patient for the presence of the polypeptides in the group consisting of SEQ ID NOs 1 to 16, wherein significantly increase levels of the polypeptides in the group consisting of SEQ ID NOs 1 to 16 is indicative of an increased likelihood that the patient will respond to treatment with the muscle anabolic agent and the absence of significantly increased levels of the polypeptides in the group consisting of SEQ ID NOs 1 to 16 is indicative of a decreased likelihood that the patient will respond to treatment with the muscle anabolic agent.

22) A kit for use in treating a patient having muscle atrophy comprising,
a) a therapeutically effective amount of a muscle anabolic agent;
b) at least one probe capable of detecting the presence of the polypeptides in the group consisting of SEQ ID NOs 1 to 16;
c) instructions for using the probe to assay a biological sample from the patient for significantly increased levels of the polypeptides in the group consisting of SEQ ID NOs 1 to 16,
d) instructions for administering the muscle anabolic agent to the patient if the biological sample from the patient has significantly increased levels of the polypeptides in the group consisting of SEQ ID NOs 1 to 16; and
e) optionally, means for administering the muscle anabolic agent to the patient.

23) The method according to claim 1, wherein muscle atrophy is selected from the group consisting of disuse atrophy, cachexia, sporadic inclusion body myositis, renal failure, AIDS (acquired immunodeficiency syndrome) and COPD (chronic obstructive pulmonary disease).

24) The method according to claim 23, wherein the cachexia is cancer cachexia.

25) The method according to claim 24, wherein the cancer is gastrointestinal cancer, pancreatic cancer, or lung cancer.

26) The method according to claim 23, wherein the patient is selected on the basis of having a significant increased level of at least 2, 3, 4, 5, 6, 7, 8, 9 or 10 of the polypeptides in the group consisting of SEQ ID NOs 1 to 16, in urine.

27) The method according to claim 23, wherein significantly increase levels of the polypeptides in the group consisting of SEQ ID NOs 1 to 16, in urine, is at least 50% above, at least 60% above, at least 70% above, at least 80% above, at least 90% above, at least 100% above, at least 110% above, at least 120% above, at least 130% above, at least 140% above, at least 150% above, at least 160% above, at least 170% above, at least 180% above, at least 190% above, or at least 200% above a standardized range of values.

28) The method according to claim 23, wherein the muscle anabolic agent is an ActRIIB antibody, such as Bimagrumab, an ActRIIA antibody, a soluble ActRIIB decoy mimetic, an anti-myostatin antibody, a myostatin propeptide, a myostatin decoy protein that binds ActRIIB but does not activate it, a beta 2 agonist, a Ghrelin agonist, an IGF-1 protein or a mimetic thereof a SARM, GH agonists/mimetics or follistatin.
